(19) United States
(12) Patent Application Publication
(10) Pub. No.: US 2012/0298882 A1
(43) Pub. Date: Nov. 29, 2012

(54) ASSEMBLY FOR DISINFECTING WATER FOR SANITARY FITTINGS

(76) Inventor: Gastone Vitali, Civita Castellana (IT)

(21) Appl. No.: 13/576,102

(22) PCT Filed: Dec. 15, 2010
(86) PCT No.: PCT/IB2010/003257

§ 371 Date: Jul. 30, 2012

(30) Foreign Application Priority Data
Jan. 29, 2010 (IT) RM2010A000033

(51) Int. Cl. C02F 1/32

(52) U.S. Cl. 250/436

(57) ABSTRACT

An assembly for disinfecting water for sanitary fittings comprising at least one container, which is comprised in an assembly for delivering or draining water and present within which is water to be disinfected. The disinfection assembly comprises a UV lamp housed inside the container and designed to irradiate the water inside the container itself.

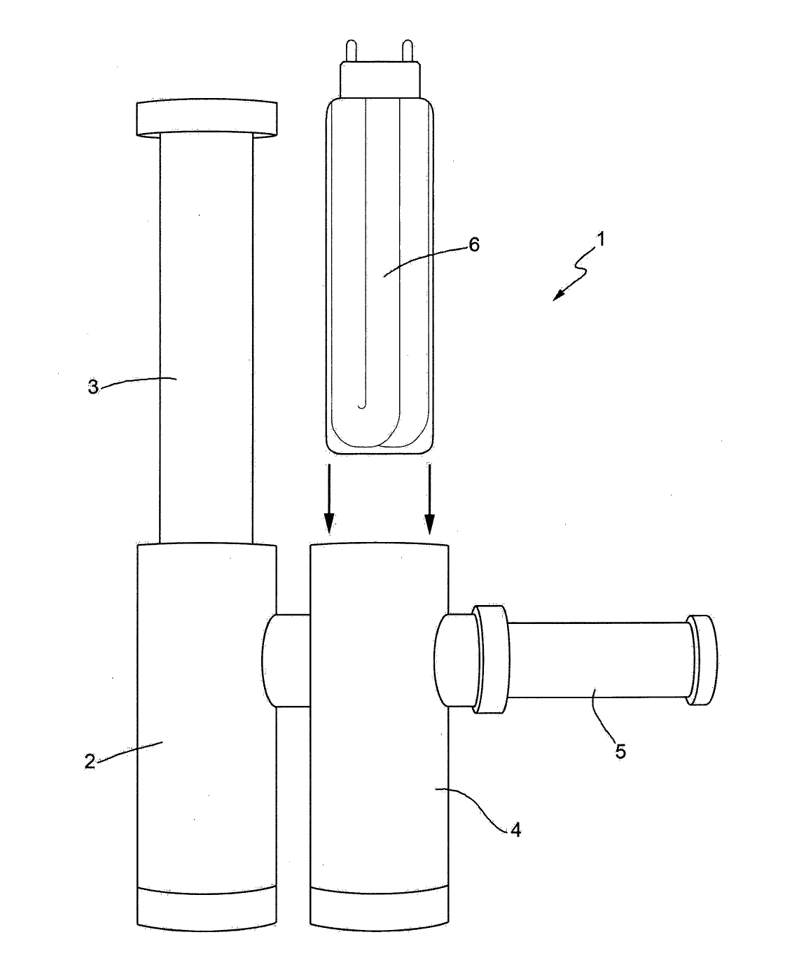

### TECHNICAL FIELD

The present invention relates to an assembly for disinfecting water for sanitary fittings.

In particular, the present invention regards a sterilizing draining assembly to which the ensuing description will make explicit reference without this implying any loss in generality.

### BACKGROUND ART

In many environments, in particular those in which sanitary activities are carried on, the problem of environmental disinfection is particularly felt on account of the obvious problems that infections may cause. For this purpose considerable economic resources are constantly invested to be able to guarantee an increasingly higher level of sterilization.

One of the possible causes of infections can derive from the return of viruses and bacteria from the sewage piping to the drain of the sanitary fitting.

Another of the possible causes of infections may regard stagnation of water at the spray heads of showers and in the delivery terminals. In these areas, for example, the legionella bacterium finds an environment favourable for its proliferation.

### DISCLOSURE OF INVENTION

The aim of the present invention is to provide an assembly for disinfecting water for sanitary fittings the technical characteristics of which are such as to prevent possible causes of infections.

The subject of the present invention is an assembly for disinfecting water for sanitary fittings the basic characteristics of which are specified in Claim 1 and preferred and/or auxiliary characteristics of which are specified in Claims 2-9.

### BRIEF DESCRIPTION OF THE DRAWING

For a better understanding of the invention, embodiments are provided hereinafter purely by way of illustrative and non-limiting example with the aid of the FIGURE of the annexed drawing, which is a side view of the disinfection assembly according to the present invention with parts separated.

### BEST MODE FOR CARRYING OUT THE INVENTION

In the FIGURE designated as a whole by 1 is a disinfection assembly according to the present invention.

The disinfection assembly 1 comprises a service siphon 2, a first pipe 3 set vertically and designed to connect a drain of a sanitary fitting (known and not illustrated for reasons of simplicity) to the siphon 2, a sterilizing siphon 4 connected to the outlet of the siphon 2, and a second pipe 5 connected to the sterilizing siphon 4 for conveying the waste water towards a sewage system.

The sterilizing siphon 4 comprises a UV lamp 6 immersed in the waste water, which has a bactericidal action and is arranged to irradiate an internal portion of the sterilizing siphon 4 itself in such a way as to ensure that the bactericidal action of the UV radiation takes place in the water contained therein.

In particular, the UV lamp 6 is a low-pressure lamp in such a way as to increase the effectiveness of the bactericidal action up to a percentage of success of 99.9%.

As is known, when a microorganism is exposed to UV radiation, the nuclei of the cells undergo modifications due to photolytic processes. Said modifications lead to blocking of cell division and reproduction with consequent elimination of the micro-organism.

Both of the pipes 3 and 5 are lined with a layer of silicone, which hence will be in direct contact with the waste water.

The silicone layer has the function of hindering bacterial proliferation, thus providing a further barrier to the return of the micro-organisms from the sewage system to the drain.

In particular, the silicone layer is made entirely of dimethyl siloxane.

Also the siphons 2 comprise a silicone lining as described for the pipes 3 and 5. In particular, all the internal walls of the siphon which in use are in contact with the water are lined with a layer of silicone.

In addition, the presence of the double siphon in series renders return of water to the drain of the sanitary fitting to which the draining assembly 1 is applied extremely difficult.

A further embodiment of the disinfection assembly of the present invention finds advantageous application when applied to a shower spray head. In this case, the UV lamp is inserted within a terminal element, made preferably of steel and set directly upstream of the shower spray head. In this way, the UV lamp of the type described above is arranged so as to irradiate all the water that traverses the terminal element to guarantee a germicidal effect on the water itself.

As has been said above as regards the siphons of the draining assembly, also in this case the internal walls of the terminal element, which in use are in contact with the water, are lined with a layer of silicone.

According to a preferred embodiment, the disinfection assembly of the present invention is comprised in a sanitary fitting both at the level of delivery of the water, such as, for example, the shower spray head, and at the level of drainage as illustrated above.

In this way, in addition to preventing return into the drain of infectious agents coming from the sewage system, sterilization of the water supplied by the sanitary fitting is also guaranteed.

As emerges from the foregoing description, the disinfection assembly forming the subject of the present invention presents technical characteristics such as to guarantee that micro-organisms of infectious agents will be unable to proliferate so that they cannot be transported by the water supplied and cannot return from the sewage systems to the drains of the sanitary fittings. In this way, any possible cause of infections is prevented. Obviously, the present invention can find interesting use above all in structures where the environment is more at risk of infections, such as for example hospitals, surgeries, and laboratories.

1. An assembly for disinfecting water for sanitary fittings, comprising at least one container, which is comprised in an assembly for delivering or draining water and within water to be disinfected is present; said disinfection assembly being characterized in that it comprises a UV lamp housed inside said container and designed to irradiate the water present inside said container.

2. The disinfection assembly according to claim 1, characterized in that said container is comprised in a water-supply assembly and consists in a terminal element set directly upstream of the spray head of a shower.

3. The disinfection assembly according to claim 1, characterized in that said container is a sterilization siphon of a draining assembly comprising a second siphon, a first pipe designed to connect a drain of a sanitary fitting to said second siphon, and a second pipe designed to drain the waste water from said siphon to a sewage system; said sterilization siphon being set between said second siphon and said second pipe.

4. The disinfection assembly according to claim 1, characterized in that said UV lamp emits in a range of wavelengths comprised between 240 and 280 nm.

5. The disinfection assembly according to claim 1, characterized in that said UV lamp emits at a radiating intensity comprised between 9 and 13 mW/cm².

6. The disinfection assembly according to claim 5, characterized in that said UV lamp is a low-pressure lamp.

7. The disinfection assembly according to claim 2, characterized in that said terminal element comprises a layer of lining, which is designed to be in contact with the water and is made of silicone.

8. The disinfection assembly according to claim 3, characterized in that said pipes and said siphons comprise a lining layer which is designed to be in contact with the waste water and is made of silicone.

9. The disinfection assembly according to claim 7, characterized in that said lining layer is made entirely of dimethyl siloxane.

10. A sanitary fitting, characterized in that it comprises an assembly according to claim 1, wherein said container is comprised in a water supply assembly and consists in a terminal element set directly upstream of the spray head of a shower, and/or is a sterilization siphon of a draining assembly comprising a second siphon, a first pipe designed to connect a drain of a sanitary fitting to said second siphon, and a second pipe designed to drain the waste water from said siphon designed to drain the waste water from said siphon to a sewage system; said sterilization siphon being set between said second siphon and said second pipe.

11. The disinfection assembly according to claim 8, characterized in that said lining layer is made entirely of dimethyl siloxane.
